(19) United States
(12) Patent Application Publication
(10) Pub. No.: US 2014/0141284 A1
(43) Pub. Date: May 22, 2014

(54) LUBRICANT COMPOSITIONS

(71) Applicants: Jiping Yang, San Jose, CA (US); Michael Joseph Stirniman, Fremont, CA (US)

(72) Inventors: Jiping Yang, San Jose, CA (US); Michael Joseph Stirniman, Fremont, CA (US)

(73) Assignee: SEAGATE TECHNOLOGY LLC, Cupertino, CA (US)

(21) Appl. No.: 14/114,923

(22) PCT Filed: Jun. 7, 2011
(86) PCT No.: PCT/US2011/039411

§ 371 Date: Jan. 14, 2014

(51) Int. Cl. G11B 5/725; C07C 49/175

(52) U.S. Cl. CPC G11B 5/725; C07C 49/175
USPC 428/833; 568/413

(57) ABSTRACT

A compound of Formula (I) includes: [L]₁-[N]ₙ—OCH₂CF₂O(CF₂CF₂O)ₚ(CF₂O)_{q}CF₂CH₂O—[M]ₘ, where L is selected from the group consisting of (AA) M is selected from the group consisting of (BB) and N is (CC) where R¹, R², and R³ may be the same or different, and are selected from the group consisting of hydroxyl, phenyl, piperonyl, carboxylic acid, amide, 2,3-di-hydroxy-1-propoxyl, acetamide, methacrylate, methyl methacrylate and glycidyl ether; and where 1=0, 1, or 2, m=0, 1, or 2, n=1 or 2, and the ratio of p to q is from about 0.1 to 3.0.

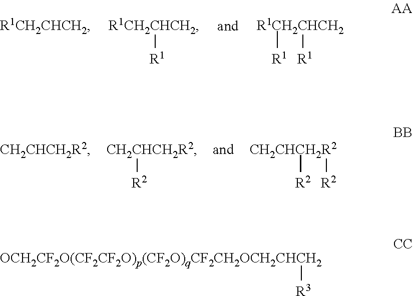

### BACKGROUND

1. Field

The present disclosure relates generally to lubricant compositions.

2. Background

High performance lubricants are used for a large number of diverse applications. The requirements of these lubricants are becoming more demanding due to a variety of factors, including miniaturization of electronic and mechanical devices, use of high temperature operating conditions, an increased expectation of product lifetimes, and an expanding range of operating and storage environments.

One application in which high performance lubricants are subject to ever increasing demands is hard disk drive magnetic recording systems. Conventional hard disk drive lubricants are typically perfluoropolyethers (PFPE's). There remains, however, a definite need for improved PFPE lubricants to meet such ever increasing demands.

### SUMMARY

In one aspect of the present disclosure, a compound of Formula I includes

[L]₁-[N]ₙ—OCH₂CF₂O(CF₂CF₂O)ₚ(CF₂O)_{q}CF₂CH₂O—[M]ₘ Formula 1

where
L is selected from the group consisting of

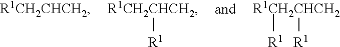

M is selected from the group consisting of

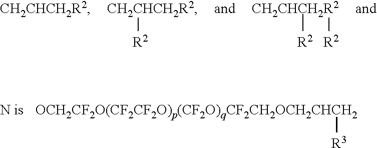

where R¹, R², and R³ may be the same or different, and are selected from the group consisting of hydroxyl, phenyl, piperonyl, carboxylic acid, amide, 2,3-dihydroxy-1-propoxyl, acetamide, methacrylate, methyl methacrylate and glycidyl ether; and where
1=0, 1, or 2,
m=0, 1, or 2,
n=1 or 2, and
the ratio of p to q is from about 0.1 to 3.0, preferably from 0.5 to 1.5, more preferably from 0.8 to 1.2.

In another aspect of the present disclosure, a compound of Formula 2 includes

[(HO)CH₂C(OH)HCH₂]—[OCH₂CF₂O(CF₂CF₂O)ₚ(CF₂O)_{q}CF₂CH₂OCH₂C(OH)HCH₂]—OCH₂CF₂O(CF₂CF₂O)ₚ(CF₂O)_{q}CF₂CH₂O—[CH₂C(OH)HCH₂(OH)] Formula 2

the ratio of p to q is from about 0.1 to 3.0, in an embodiment from 0.5 to 1.5, in another embodiment from 0.8 to 1.2.

In yet another aspect of the present disclosure, an apparatus includes a substrate; a magnetic layer for recording information disposed on the substrate; a protective overcoat for protecting said magnetic layer; and a lubricant. The lubricant is a compound of Formula 1

[L]₁-[N]ₙ—OCH₂CF₂O(CF₂CF₂O)ₚ(CF₂O)_{q}CF₂CH₂O—[M] Formula 1

where
L is selected from the group consisting of

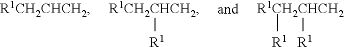

M is selected from the group consisting of

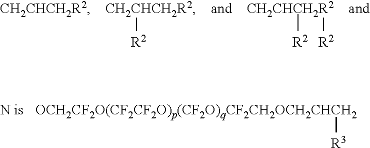

where R¹, R², and R³ may be the same or different, and are selected from the group consisting of hydroxyl, phenyl, piperonyl, carboxylic acid, amide, 2,3-dihydroxy-1-propoxyl, acetamide, methacrylate, methyl methacrylate and glycidyl ether; and where
1=0, 1, or 2,
m=0, 1, or 2,
n=1 or 2,
the ratio of p to q is from about 0.1 to 3.0, preferably from 0.5 to 1.5, more preferably from 0.8 to 1.2.

In a further aspect of the present disclosure, an apparatus includes a substrate; a magnetic layer for recording information disposed on the substrate; a protective overcoat for protecting said magnetic layer; and a lubricant. The lubricant is a compound of Formula 2

[(HO)CH₂C(OH)HCH₂]—[OCH₂CF₂O(CF₂CF₂O)ₚ(CF₂O)_{q}CF₂CH₂OCH₂C(OH)HCH₂]—OCH₂CF₂O(CF₂CF₂O)ₚ(CF₂O)_{q}CF₂CH₂O—[CH₂C(OH)HCH₂(OH)] Formula 2

the ratio of p to q is from about 0.1 to 3.0, preferably from 0.5 to 1.5, more preferably from 0.8 to 1.2.

### BRIEF DESCRIPTION OF THE DRAWINGS

FIG. 1 is a block diagram showing an example of a recording media structure including a lubricant layer.

FIGS. 2A and 2B are diagrams comparing the distance between the flying head and the lubricant surface when using (A) conventional lubricant compositions in the lubricant layer and (B) the lubricant composition embodiments of the present disclosure.

FIG. 3 shows an example of a chemical structure of a composition that may be used as a lubricant.

FIG. 4 is a box plot comparing the clearance of the composition shown in FIG. 3 with another lubricant composition (Z Tetraol 2700GT).

FIG. 5 is a graph comparing the TOC pick-up of the composition shown in FIG. 3 to the TOC pick-up of another lubricant composition (Z Tetraol 2700GT).

### DETAILED DESCRIPTION

Various concepts are described more fully hereinafter with reference to the accompanying drawings. These concepts, however, may be embodied in many different forms and should not be construed as being limited by any specific structure or process presented in this disclosure. Rather, the specific details presented throughout this disclosure are provided so that the disclosure will be thorough and complete, and will fully convey the scope of these concepts to those skilled in the art. However, it will be apparent to those skilled in the art that the various concepts presented in this disclosure may be practiced without these specific details. In some instances, well-known aspects of the disclosure may be shown in block diagram form in order to avoid obscuring the various concepts presented throughout this disclosure.

Various aspects of compositions for lubricating storage media and storage media incorporating the same will now be presented. However, as those skilled in the art will readily appreciate, these aspects may be extended to other compositions and apparatus.

The compositions comprise a polyfluoropolyether (PFPE) backbone having one or more functional groups provided on each end of the backbone, and one or more functional groups provided on the backbone between the ends. The functional groups may cause the lubricant molecule to bond to and lie flat on the carbon overcoat. The low profile structure of the lubricant coating results in optimized tribology performance, and permits reduced HMS.

### 1. DEFINITIONS

All scientific and technical terms used in this application have meanings commonly used in the art unless otherwise specified. As used in this application, the following words or phrases have the meanings specified.

As used herein “perfluoropolyether” or “PFPE” lubricant means long chain polymers composed of repeat units of small perfluorinated aliphatic oxides such as perfluoroethylene oxide or perfluoropropylene oxide. Examples of commercially available PFPE polymers include, but are not limited to, Fomblin Z (random copolymer of CF₂CF₂O and CF₂O units) and Fomblin Y (random copolymer of CF(CF₃)CF₂O and CF₂O) and their functional derivates such as ZDOL, ZDOL TX, and ZTETRAOL (available from Montedison), Demnum (a homopolymer of CF₂CF₂CF₂O; available from Daikin), and Krytox (a homopolymer of CF(CF₃)CF₂O).

As used herein, “storage medium” or “storage media” means any apparatus on which information can be stored. The storage medium may be used in a computer disk drive. The storage medium may be provided in the form of a thin film, which may be magnetic, and may be formed, for example, by applying a cobalt, platinum, and/or chromium alloy film over a supporting substrate. One example of a supporting substrate is a nickel-phosphorous plated aluminum, which may be coated with a chromium underlayer. The storage medium may also have a protective layer applied over the magnetic layer. Examples of protective overcoats include, but are not limited to, sputtered ceramic zirconium oxide and amorphous films of silicon dioxide.

As used herein, “recording surface” or “data zone” means a portion of the magnetic disk adapted to magnetically record information. The magnetic recording may occur via a slider of a magnetic head assembly which travels over the disk surface. The magnetic head assembly may comprise an air bearing read/write head. A “data zone” is a zone where the slider flies over the disk and stores magnetic data. The recording surface or data zone can be smooth or rough. A smooth data zone may have a roughness average of less than about 15 Å, or a roughness average of less than about 10 Å.

As used herein, “landing zone” means a zone where the slider rests while the disk drive is off, and takes off from when the disk drive is started up.

As used herein, “backbone” means the main chain of a PFPE composition. The backbone portion of the composition does not bind to a substrate, such as the DLC coating layer. Elements of the backbone may include carbon (C), nitrogen (N), oxygen (O), or other linker elements. The PFPE compositions may include two or more backbones attached at an anchor point or a plurality of anchor points.

As used herein, “functional group” means a substituent attached to the PFPE backbone that is capable of interacting with a surface to be lubricated. Examples of functional groups that may be used in the disclosed lubricants include hydroxyl, phenyl, piperonyl, carboxylic acid, amide, 2,3-dihydroxy-1-propoxyl, acetamide, methacrylate, methyl methacrylate and glycidyl ether. Functional groups may cause the PFPE compositions to attach to a surface through polar interactions, and generally include the property of adhering to a surface. If the bonding enhancer is methacrylate, methyl methacrylate or glycidyl ether, then ultraviolet light can optionally be used to activate the bonding enhancer.

As used herein, “terminal” means a functional group that is attached to a linker element of the backbone that is on the end of the backbone, or it is attached to a linker element of the backbone that is in turn attached to a linker element of the backbone that has a terminal functional group attached.

As used herein, “non-terminal” means a functional group that is attached to a linker element of the backbone that is not at the end of the backbone, and is not attached to a linker element of the backbone that has a terminal functional group attached. A non-terminal functional group may be attached at any point or points along the backbone that are not terminal. One or more non-terminal functional groups may be provided in order to reduce the amount that the backbone extends from the surface.

### 2. COMPOSITIONS

The lubricant compositions include molecules that comprise a PFPE backbone, where each end of the PFPE backbone terminates with one or more functional groups. One or more non-terminal functional groups are also attached to the PFPE backbone. The non-terminal functional group, which may be provided around or near the center of the PFPE backbone, causes the lubricant molecule to lie flat on carbon overcoat while the terminal groups provided at the ends of the PFPE backbone provide strong bonding at the ends of the molecular chains. In addition to improving bonding of the composition to the carbon overcoat, the use of the functional groups in this configuration beneficially reduces the distance that the composition extends up from the carbon overcoat into the HMS.

The compositions may have the structure shown in Formula I

[L]₁-[N]ₙ—OCH₂CF₂O(CF₂CF₂O)ₚ(CF₂O)_{q}CF₂CH₂O—[M]ₘ Formula 1

where
L is selected from the group consisting of

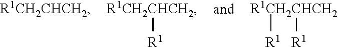

M is selected from the group consisting of

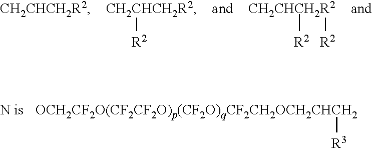

where R¹, R², and R³ may be the same or different, and are selected from the group consisting of hydroxyl, phenyl, piperonyl, carboxylic acid, amide, 2,3-dihydroxy-1-propoxyl, acetamide, methacrylate, methyl methacrylate and glycidyl ether; and where
1=0, 1, or 2,
m=0, 1, or 2,
n=1 or 2, and
the ratio of p to q is from about 0.1 to 3.0, preferably from 0.5 to 1.5, more preferably from 0.8 to 1.2.

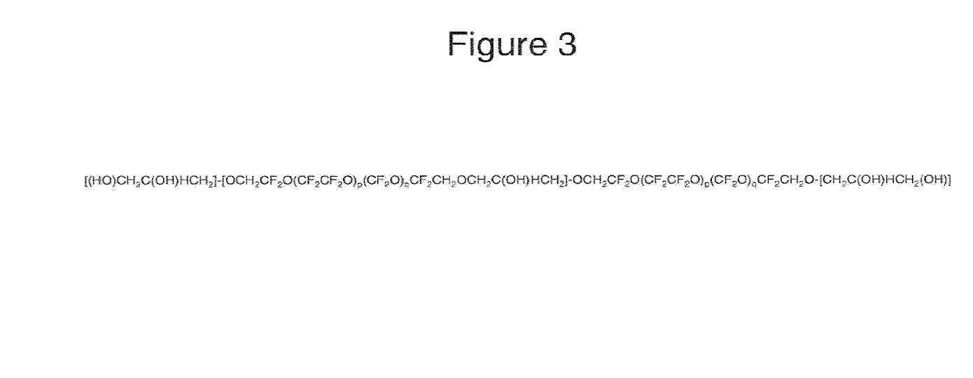

The compositions have the structure shown in FIG. 3, in which the structure of the composition is

[(HO)CH₂C(OH)HCH₂]—[OCH₂CF₂O(CF₂CF₂O)ₚ(CF₂O)_{q}CF₂CH₂OCH₂C(OH)HCH₂—OCH₂CF₂O(CF₂CF₂O)ₚ(CF₂O)_{q}CF₂CH₂O—[CH₂C(OH)HCH₂(OH)] Formula 2

where the ratio of p to q is from about 0.1 to 3.0, preferably from 0.5-1.5, more preferably from 0.8 to 1.2.

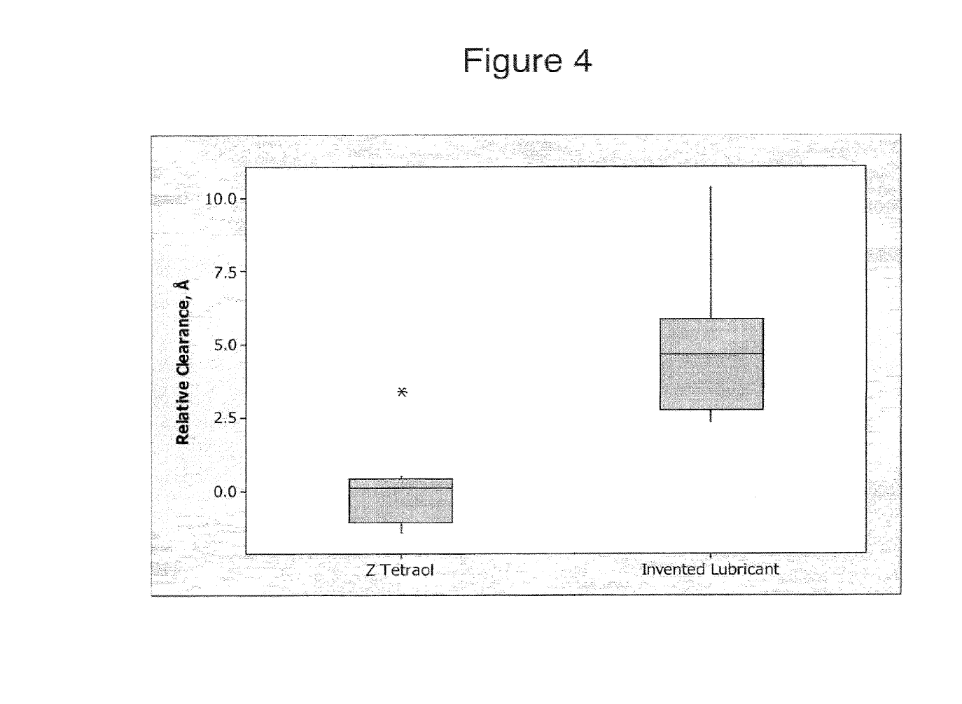

A 13C NMR spectrum provided as FIG. 4 shows the peaks associated with the carbon atoms contained in the composition of Formula 2, which is also shown in FIG. 3. The compositions also encompass compounds that may differ from the compositions of Formula 1 and Formula 2, but provide substantially the same NMR spectrum as is shown in FIG. 4.

In the compositions of Formula 1 and Formula 2, the values of p and q may be selected so as to provide compounds having a molecular weight that falls within a desired range. The number average molecular weight may range, for example, from 1000 Dalton to 5000 Dalton, more preferably from 2000 Dalton to 4000 Dalton.

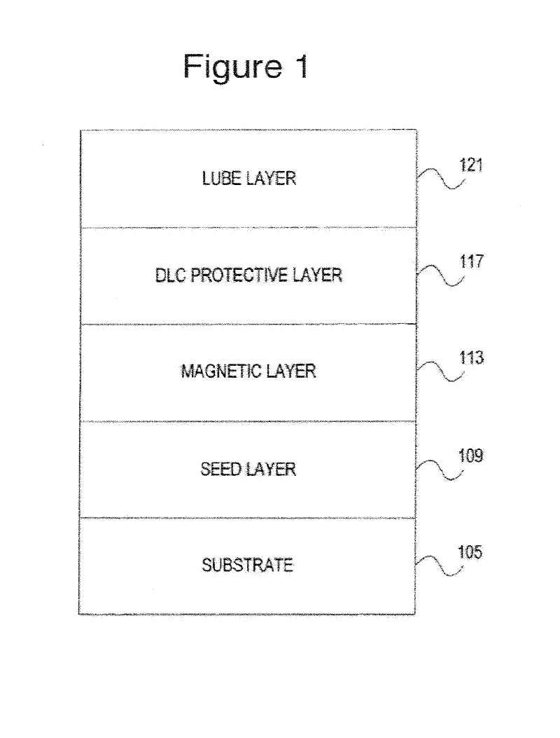

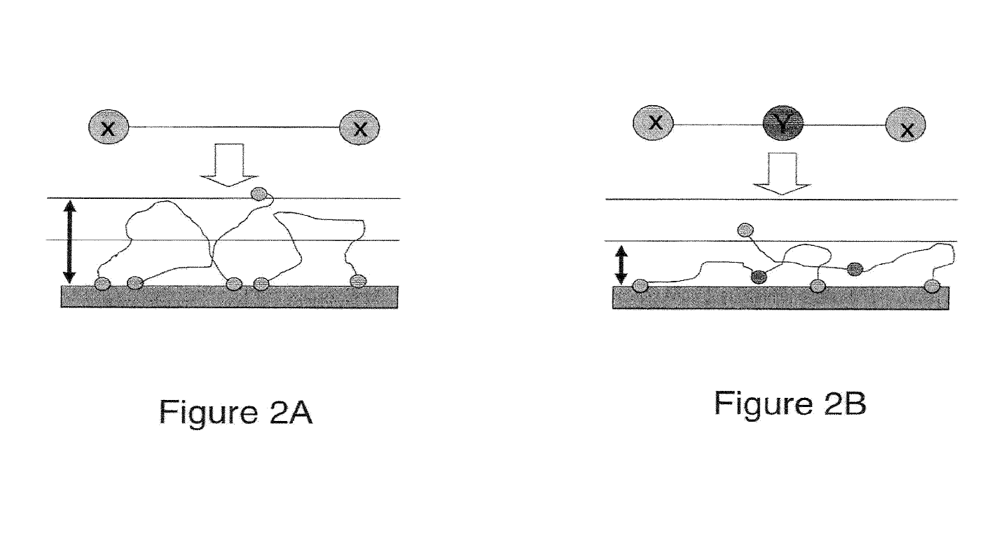

FIGS. 2A and 2B are diagrams illustrating the difference in the distance between a flying head 200 and a surface to be lubricated 202 when using a conventional lubricant composition 204 in a lubricant layer 206 (FIG. 2A) and a lubricant composition in accordance with the present disclosure 208 in the lubricant layer (FIG. 2B). The conventional lubricant comprises a backbone 210 formed of repeat units of small perfluorinated aliphatic oxides 212. The lubricant composition in accordance with the present disclosure 208 corresponds to a composition as shown in FIG. 1, where 1=0, m=0 and n=1 and includes a non-terminal functional group 214, in addition to a backbone 210 formed of repeat units of small perfluorinated aliphatic oxides 212.

The amount L₁ that the backbone 210 extends from surface to be lubricated 202 for the conventional lubricant composition 204 is significantly greater than the amount L₂ that the backbone 210 extends from the surface to be lubricated 202 for the lubricant composition in accordance with the disclosure 208, because of the interaction between the non-terminal functional group and the surface. Consequently, the distance between the flying head 200 and the surface can be significantly smaller when a conventional lubricant composition is used, than the distance between the flying head 200 and surface when a lubricant composition in accordance with the disclosure is used.

### 3. METHODS FOR LUBRICATING, AND APPARATUS INCORPORATING THE LUBRICANT

Methods of lubricating apparatus, such as recording media, may incorporate the compositions. The methods generally comprise applying the compositions to the apparatus as it is being formed. Apparatus, such as recording media including a lubricant layer formed from the compositions, are also provided.

FIG. 1 is an illustration showing the layers of a recording media structure including a substrate 105, a seed layer 109, a magnetic layer 113, a protective layer 117, and a lubricant layer 121. The initial layer of the media structure is the substrate 105, which is typically made of nickel-phosphorous plated aluminum or glass that has been textured. The seed layer 109, typically made of chromium, is a thin film that is deposited onto the substrate 105 creating an interface of intermixed substrate 105 layer molecules and seed layer 109 molecules between the two. The magnetic layer 113, typically made of a magnetic alloy containing cobalt (Co), platinum (Pt) and chromium (Cr), is a thin film deposited on top of the seed layer 109 creating a second interface of intermixed seed layer 109 molecules and magnetic layer 113 molecules between the two. The magnetic layer may be applied at a thickness of about 500 Å over the substrate.

The protective layer 117 may be a diamond like carbon (DLC) layer, which is typically made of carbon and hydrogen. The protective layer 117 is a thin film that is deposited on top of the magnetic layer 113, creating a third interface of intermixed magnetic layer 113 molecules and protective layer 117 molecules between the two. When provided, the DLC layer 117 exhibits properties between those of graphite and diamond. Thin layers of DLC are deposited on disks using a thin film deposition technique such as ion beam deposition (IBD), plasma enhanced chemical vapor deposition (PECVD), magnetron sputtering, radio frequency sputtering or chemical vapor deposition (CVD). During the deposition process, adjusting sputtering gas mixtures of argon and hydrogen varies the concentrations of hydrogen found in the DLC. Other materials that may be used for the protective layer 117 include sputtered ceramic zirconium oxide, and amorphous films of silicon dioxide. The protective layer may be about 150 Å thick, and the protective layer may be less than 100 Å thick.

Lubricant layer 121 may be deposited on top of the protective layer 117 for added protection, lubrication, and enhanced disk drive reliability. Lubricant layer 121 further reduces wear of the disk due to contact with the magnetic head assembly. The lubricant is deposited on top of the protective layer 117, thereby creating a fourth interface of intermixed protective layer 117 molecules and lubricant layer 121 molecules.

The durability and reliability of recording media is achieved primarily by the application of the protective layer 117 and the lubricant layer 121. As the thickness of the carbon overcoat and lubricant protective layers is continuously reduced, greater integration of the overcoat and lubricant is specified to provide a more durable protective film. The conformation of the lubricant molecules on the carbon overcoat is also of importance to the head-media spacing (HMS). In addition, lubricants that extend their molecular structure across the carbon surface are able to cover the entire overcoat surface better than those that tend to extend their molecular chains away from the carbon surface.

The methods for lubricating an apparatus, such as a recording media, include providing a lubricant layer on a layer to be lubricated. The lubricant layer may be formed using the compositions of Formula 1 and/or Formula 2.

The lubricant layer may be applied evenly over the recording media in a thin film having a thickness from about 5 Å to about 50 Å, or from about 8 Å to about 40 Å, or from about 10 Å to about 20 Å. The lubricant layer may be made as thin as possible, while still remaining possessing the durability and flyability used to provide functional recording media. As will be understood by those skilled in the art, the selection of the thickness of the lubricant layer may depend on interactions between the recording media and the head assembly, such as the static friction or “stiction” force on the slider, air shear, and the tendency of the lubricant composition to evaporate. The amount of lubricant on the data zone may also minimize wear and damage to the disk caused by occasional contacts between the magnetic head assembly and the disk.

The lubricant composition may be applied to either or both of the landing zone and the data zone of the recording media. The lubricant composition may be applied as an unbonded layer having a thickness of about 5 Å to about 50 Å. The lubricant composition also can be applied in a bonded layer having a thickness of about 5 Å to about 50 Å. The amount of lubricant on the landing zone may be sufficient to minimize stiction forces on the slider. The amount of lubricant on the data zone may be sufficient to minimize wear and damage to the recording media caused by occasional contacts between the magnetic head assembly and the recording media.

Apparatus incorporating the recording media are adapted to magnetically record information via an air bearing read/write head. The apparatus may be a computer disk drive. Those skilled in the art will appreciate that the lubricant composition and methods of preparing recording media using the lubricant composition can be incorporated into methods for manufacturing disks and disk drives, such as thin film magnetic disks and disk drives. In accordance with these methods, more durable, higher-density recording media may be provided.

These and other aspects of the compositions are further described in the non-limiting Examples set forth below.

### EXAMPLES

### Example 4

### Clearance Capability of Lubricated Recording Media

FIG. 4 is a box plot comparing the clearance capability of a recording medium prepared using the composition shown in FIG. 3 with the clearance capability of a recording medium prepared using another lubricant composition (Z Tetraol 2700GT). Clearance is described as the distance between lowest point of the flying head and the top of the lubricant surface. The clearance capability of the recording medium using the composition shown in FIG. 3 results in more distance between the head and lubricant surface for a given head-media spacing, reducing head-disc interactions and promoting disk drive reliability. The clearance difference in FIG. 9 was measured using an adaptive fly height head to protrude the close point of the head (keeping the flying height constant) until it touches the top of the lubricant film, with contact determined by an increase in the acoustical emission signal from a sensor on the head. The same head is used to measure the different lubricants, which keeps the flying height constant and allows measurement of the difference in clearance between the lubricants. The Z-Tetraol is arbitrarily set to zero to show the measured difference in clearance from the lubricant.

### Example 6

### TOC Pick-Up of Lubricants

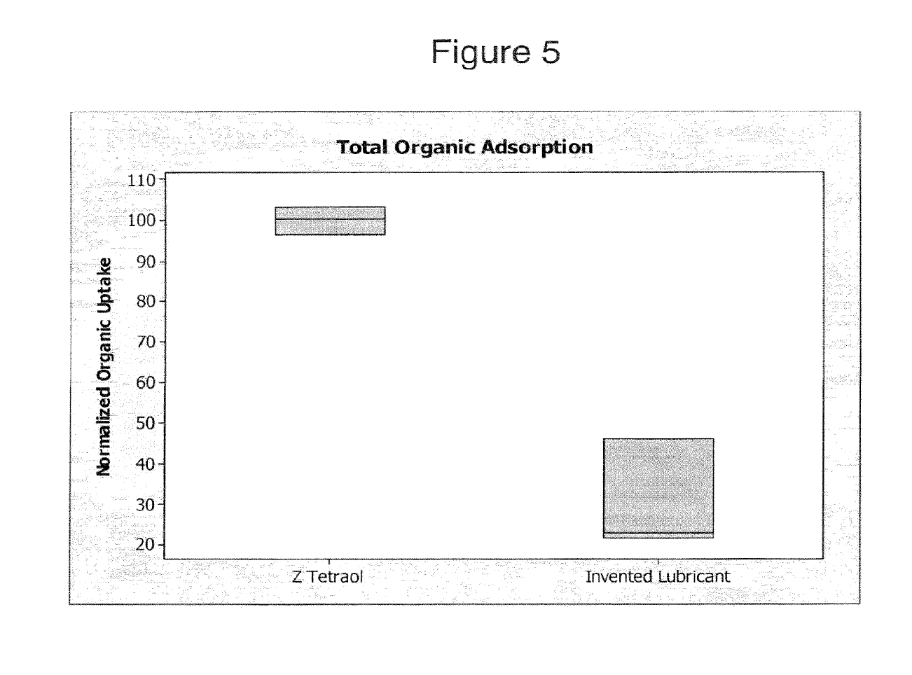

FIG. 5 is a graph comparing the TOC pick-up of the compound shown in FIG. 3 to the TOC pick-up of a lubricant composition (Z Tetraol 2700GT). Organic contaminants such as hydrocarbons can compromise head-disk interface reliability in the disk drive, by adsorption onto the media surface and subsequent accumulation onto the read-write head. One function of the lubricant is to provide an inert barrier film to block adsorption of any contaminants that may be present in the drive, e.g. due to outgassing from other drive internal components. The data in FIG. 11 were collected by exposing media coated with different lubricants to model organic contaminants at elevated temperature in a closed system, followed by extraction and quantification of the amount adsorbed. The compounds show a significant reduction in the level of adsorbed contamination as compared to the lubricant Z-Tetraol.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. A phrase referring to “at least one of” a list of items refers to any combination of those items, including single members. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

1. A compound of Formula 1, comprising:
[L]₁-[N]ₙ—OCH₂CF₂O(CF₂CF₂O)ₚ(CF₂O)_{q}CF₂CH₂O—[M]ₘ Formula 1
where
L is selected from the group consisting of 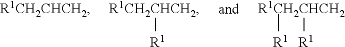
M is selected from the group consisting of 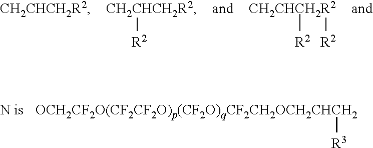
where R¹, R², and R³ may be the same or different, and are selected from the group consisting of hydroxyl, phenyl, piperonyl, carboxylic acid, amide, 2,3-dihydroxy-1-propoxyl, acetamide, methacrylate, methyl methacrylate and glycidyl ether; and
where
1=0, 1, or 2,
m=0, 1, or 2,
n=1 or 2, and
the ratio of p to q is from about 0.1 to 3.0.

2. The compound of claim 1, wherein L is

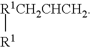

3. The compound of claim 1, wherein M is

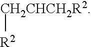

4. The compound of claim 1, wherein 1 is 1.

5. The compound of claim 1, wherein m is 1.

6. The compound of claim 1, wherein n is 1.

7. The compound of claim 1, wherein R¹ is hydroxyl.

8. The compound of claim 1, wherein R² is hydroxyl.

9. The compound of claim 1, wherein R³ is hydroxyl.

10. A compound of Formula 2,
[(HO)CH₂C(OH)HCH₂]—[OCH₂CF₂O(CF₂CF₂O)ₚ(CF₂O)_{q}CF₂CH₂OCH₂C(OH)HCH₂]—OCH₂CF₂O(CF₂CF₂O)ₚ(CF₂O)_{q}CF₂CH₂O—[CH₂C(OH)HCH₂(OH)] Formula 2
where the ratio of p to q is from about 0.1 to 3.0.

11. An apparatus, comprising:
a substrate;
a magnetic layer for recording information disposed on the substrate;
a protective overcoat for protecting said magnetic layer; and
a lubricant comprising a compound of Formula 1
[L]₁-[N]ₙ—OCH₂CF₂O(CF₂CF₂O)ₚ(CF₂O)_{q}CF₂CH₂O—[M]ₘ Formula 1
where
L is selected from the group consisting of 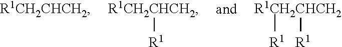
M is selected from the group consisting of 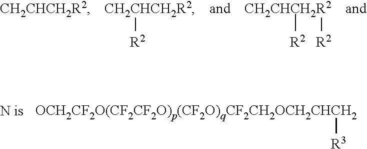
where R¹, R², and R³ may be the same or different, and are selected from the group consisting of hydroxyl, phenyl, piperonyl, carboxylic acid, amide, 2,3-dihydroxy-1-propoxyl, acetamide, methacrylate, methyl methacrylate and glycidyl ether; and
where
1=0, 1, or 2,
m=0, 1, or 2,
n=1 or 2, and
the ratio of p to q is from about 0.1 to 3.0.

12. The apparatus of claim 11, wherein L is

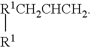

13. The apparatus of claim 11, wherein M is

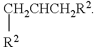

14. The apparatus of claim 1, wherein 1 is 1.

15. The apparatus of claim 1, wherein m is 1.

16. The apparatus of claim 1, wherein n is 1.

17. The apparatus of claim 1, wherein R¹ is hydroxyl.

18. The apparatus of claim 1, wherein R² is hydroxyl.

19. The apparatus of claim 1, wherein R³ is hydroxyl.

20. An apparatus, comprising:
a substrate;
a magnetic layer for recording information disposed on the substrate;
a protective overcoat for protecting said magnetic layer; and
a lubricant comprising a compound of Formula 2
[(HO)CH₂C(OH)HCH₂]—[OCH₂CF₂O(CF₂CF₂O)ₚ(CF₂O)_{q}CF₂CH₂OCH₂C(OH)HCH₂]—OCH₂CF₂O(CF₂CF₂O)ₚ(CF₂O)_{q}CF₂CH₂O—[CH₂C(OH)HCH₂(OH)] Formula 2
where the ratio of p to q is from about 0.1 to 3.0.
